(19) United States
(12) Patent Application Publication
(10) Pub. No.: US 2024/0279289 A1
(43) Pub. Date: Aug. 22, 2024

(54) METHODS OF RECOMBINANT PRODUCTION OF LECTINS

(71) Applicant: Phyto42, Las Vegas, NV (US)

(72) Inventors: Kevin Lau, San Jose, CA (US); De-Yu Xie, Cary, NC (US); Lawrence Chan, Sunnyvale, CA (US)

(21) Appl. No.: 18/421,896

(22) Filed: Jan. 24, 2024

Related U.S. Application Data
(60) Provisional application No. 63/446,407, filed on Feb. 17, 2023.

(51) Int. Cl. C07K 14/42; C07K 1/22; C12N 15/70; C12P 21/02

(52) U.S. Cl. CPC C07K 14/42; C07K 1/22; C12N 15/70; C12P 21/02; C12N 2800/101

(57) ABSTRACT

A method for producing a recombinant mannose binding lectin for pattern recognition-based innate immune response targeting and activation against glycosylated pathogens. The method of recombinant mannose binding lectin production includes an inducible recombinant expression system and multi-stage purification.

### RELATED APPLICATION

This application claims the benefit under 35 U.S.C. § 119(e) of U.S. Provisional Application No. 63/446,407, filed Feb. 17, 2023, entire contents of which are hereby incorporated herein by reference for all purposes as if fully set forth herein.

This application is related to U.S. Provisional Application No. 63/402,278, filed Aug. 30, 2022, and U.S. patent application Ser. No. 18/238,641, filed Aug. 28, 2023, entire contents of which are hereby incorporated herein by reference for all purposes as if fully set forth herein.

### TECHNICAL FIELD

The invention relates to methods of recombinant production of lectins. Specifically, the invention relates to methods for producing a recombinant mannose binding lectin with recombinant expression and purification of a tagged lectin.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

The Severe Acute Respiratory Syndrome CoronaVirus 2 (SARS-CoV-2) virus is responsible for the global COVID-19 pandemic, which continues to spread. Public health measures in the USA and other countries have had limited efficacy with masks and social distancing as the primary methods for reducing infection rates by presenting physical barriers to reduce exposure and lower viral loads.

Vaccines, which train the human immune system to provide an acquired immunity, are another method for reducing infection rates. COVID-19 vaccines help protect against the illness by contributing to adaptive immunity and training our adaptive immune system to produce relevant antibodies. In particular, vaccines attempt to train our adaptive immune system to produce antibodies that target exposed portions of pathogens, including, for example, the spike protein of SARS coronaviruses. But, the SARS spike protein is continuously mutating, and it can be difficult for vaccines to keep up with these mutations. And, vaccines have had some difficulty in protecting against long COVID or secondary effects. Modern vaccines are also inaccessible to many due to their high cost. Even if vaccines were accessible to everyone, vaccine efficacy has been shown to wane over time, which means that frequent boosters, possibly matched to the current strains, are needed. There already exists vaccine hesitancy among the general population, and those who are fully vaccinated are becoming reluctant, for various reasons, to receive periodic boosters, especially with the frequency of break-through infections.

Applicant has made an unexpected discovery that certain plant lectins are capable of inhibiting the SARS-CoV-2 virus and developed a composition that includes a certain plant lectin, to mitigate COVID-19 risks, as described in co-pending U.S. patent application Ser. No. 18/238,641, filed Aug. 28, 2023, entire contents of which are hereby incorporated herein by reference for all purposes as if fully set forth herein. While such lectins may be isolated and characterized from plants, its low concentration makes its application in an industry as massive as healthcare infeasible. Accordingly, there is an urgent need for a recombinant production of such lectins that allows for fast, high volume production.

### SUMMARY

The appended claims may serve as a summary of the invention.

### BRIEF DESCRIPTION OF THE FIGURES

The present invention is illustrated by way of example, and not by way of limitation, in the figures of the accompanying drawings and in which like reference numerals refer to similar elements and in which:

FIG. 1 illustrates MBL binding and complement activation.

FIG. 2 illustrates a flow diagram of an immune system activation process.

FIG. 3 illustrates an overview of the recombinant MBL production process.

FIG. 4 illustrates an example recombinant lectin expression process flow.

FIG. 5 illustrates a pET32a-Trx-Lec vector for recombinant lectin expression.

FIG. 6 illustrates a pDEST15-GST-Lec vector for recombinant lectin expression.

FIG. 7 illustrates a recombinant lectin purification process.

FIG. 8 illustrates a multi-step purification process using an affinity tag and acetone precipitation.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

Embodiments are described herein in sections according to the following outline:
1. GENERAL OVERVIEW
2. HUMAN IMMUNE SYSTEM INTRODUCTION
3. MANNOSE-BINDING LECTINS
4. rMBL EXPRESSION
5. rMBL PURIFICATION
6. EXAMPLE PURIFICATION PROCESSES
7. OTHER ASPECTS OF DISCLOSURE

### 1. General Overview

The present disclosure is based, in part, on the unexpected discovery that certain lectins mitigate COVID-19 risks. In particular, these certain lectins are capable of inhibiting the Severe Acute Respiratory Syndrome CoronaVirus 2 (SARS-CoV-2) virus. For example, Applicant has found several plant lectins that have high affinity for SARS-CoV-2 and isolated one of those, allium porrum agglutinin (APA), with the highest affinity from allium porrum. Techniques disclosed herein produce recombinant APA (rAPA) lectin, in fast and high volumes, for therapeutic use which enables an innate immune response against SARS-CoV-2 which is expected to reduce infection risk and improve outcomes.

The rAPA lectin enables molecular pattern recognition based targeting of one or more infectious agents, including SARS coronaviruses, and activating an innate immune response as part of a composition as described in U.S. Provisional Application No. 63/402,278, filed Aug. 30, 2022, and U.S. patent application Ser. No. 18/238,641, filed Aug. 28, 2023, entire contents of which are hereby incorporated herein by reference for all purposes as if fully set forth herein. An example composition, as described in U.S. patent application Ser. No. 18/238,641, includes a lectin component (e.g., APA, etc.), a pharmaceutical component (e.g., saline, etc.), a flavonoid component (e.g., Kaempferol and/or EGCG, etc.), and an optional supportive component (e.g., calcium, etc.).

Lectins may be classified, for example, by their affinity where an example lectin may be one of a mannose-binding lectin (MBL), a N-acetyl glucosamine-binding lectin, or a jacalin-binding lectin. Lectin classification continues to evolve and it's important to note that their classification does not imply any limits to the lectin binding affinity. For example, a mannose-binding lectin may also have high binding affinity to N-acetyl glucosamine and an N-acetyl glucosamine lectin may also have high binding affinity for mannose. As a result, the terms mannose-binding lectin and MBL may be interpreted as either the classification and/or a lectins' ability to bind to high-mannose or fucose structures as appropriate. APA may be classified as a MBL, and rAPA may be classified as a rMBL.

Lectins are proteins that may form and/or re-form into one of many structures including, most commonly: a monomer structure, a dimer structure, a trimer structure, or a tetramer structure. A lectin consisting of a single protein strand is known as a monomer. When a lectin is composed of multiple strands (e.g., dimer, trimer, tetramer, etc.), the strands may be the same or different which results in a multitude of possible lectin forms. When the strands are the same, the homo-prefix may be used to describe a lectin (e.g., homodimer, homotrimer, homotetramer, etc.) When the strands are different, the hetero-prefix may be used to describe a lectin (e.g., heterodimer, heterotrimer, heterotetramer, etc.). The structure of a lectin defines its binding affinity and binding specificity which are key factors determining the amount of lectin necessary to be effective against a glycosylated pathogen. The rAPA is a homotrimer consisting of three identical strands.

### 2. Human Immune System Introduction

The human immune system is a complex combination of organs, cells, proteins and tissues, which work together to protect the human body from invading pathogens. Our immune system consists of two parts: an innate immune system and an adaptive immune system.

Innate immunity is considered a general or “nonspecific” defense system. Our innate immune system (which includes our skin, mucous membranes, phagocytes, natural killer cells, and various proteins and enzymes) is our first line of defense against invading pathogens. The purpose of the innate immune response is to immediately prevent the spread and movement of invading pathogens throughout the human body by blocking their entry and generating rapid inflammatory responses in response to signals from molecular pattern recognition receptors (PRRs). The innate immune system is constantly protecting us from infection by pathogens via pathogen recognition and elimination.

Adaptive immunity is our second line of defense against invading pathogens. If invading pathogens evade our innate immune system, then our adaptive immune system is activated. Our adaptive immune system builds antibodies through, for example, exposure to diseases or vaccinations. Vaccines train our adaptive immune system to provide an acquired immunity, which is activated after pathogens evade our innate immune system.

### 3. Mannose-Binding Lectins

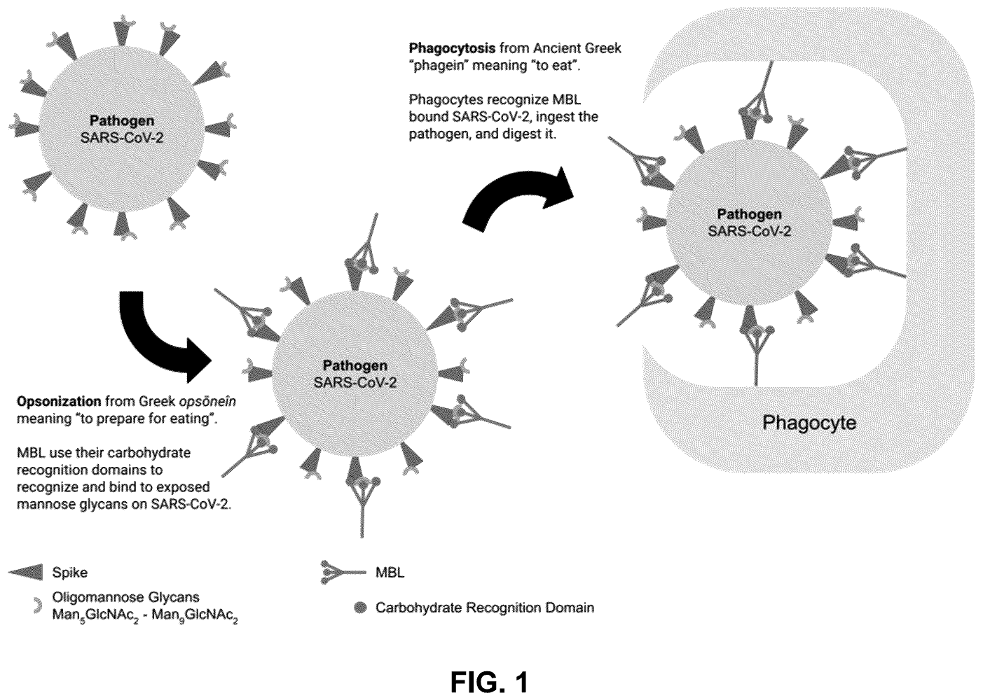

One part of the innate immune system includes lectins, specifically mannose binding lectins (MBLs), which are proteins that help our innate immune system recognize pathogens. Lectins, particularly from certain plants, are commonly identified as an “anti-nutrient” due to their association with various negative health effects and conditions. MBLs are proteins produced by the liver and secreted into the serum where they can activate an immune response. MBLs have carbohydrate recognition domains for prototypical pattern recognition allowing them to recognize and bind to infectious agents, including bacteria and viruses, and activating the innate immune system against recognized pathogens. The carbohydrate recognition domains on MBLs recognize pathogen-associated molecular patterns (PAMPs) to identify pathogens for the innate immune system to destroy. MBL binding and complement activation enhances phagocytosis by acting as an opsonin using a lectin pathway, as illustrated in FIG. 1. Opsonization leads to phagocytosis where the bound pathogens are destroyed. Complement activation further serves as a bridge to the adaptive immune system for producing antigen-specific antibodies, possibly resulting in acquired immunity.

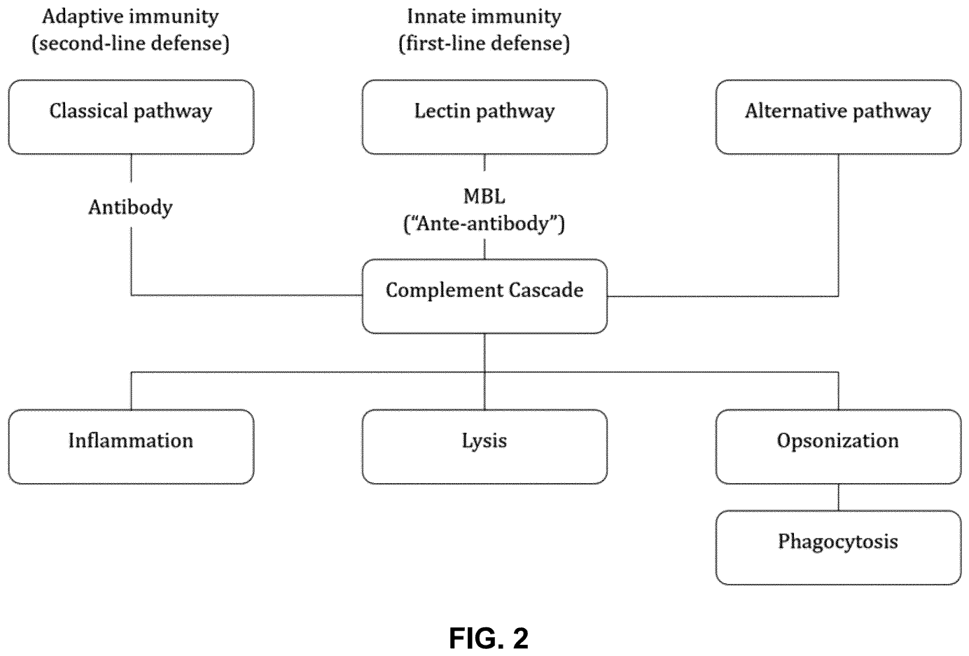

FIG. 2 illustrates a flow diagram of an immune system activation process. As part of our innate immune system, MBLs function as part of our first line of defense against invading pathogens. Conceptually, MBLs may be considered as an “ante-antibody” because of its defensive role at recognizing pathogens during the initial delay period required to develop an antibody response.

As illustrated in FIG. 2, MBLs (lectin pathway) and antibodies (classical pathway) activate parallel pathways for initiating the opsonization pathway of complement, important in tagging and removal of foreign bodies such as invading pathogens, e.g., by phagocytosis. Unlike vaccines, which require days to train the adaptive immune response and days to produce antibodies, the innate immune system is always active. As part of our innate immune system, MBLs can respond to pathogens immediately, enabling therapeutic and preventative applications.

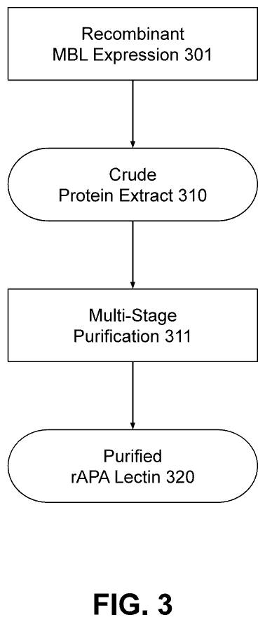

FIG. 3 illustrates an overview of a recombinant production process for rAPA. The recombinant product process for rAPA includes a recombinant MBL expression step 301 to yield crude protein extract 310 and a multi-stage purification step 311 to yield purified rAPA lectin 320. Each of the steps of FIG. 3 are further described below.

### 4. rMBL Expression

Proteins are synthesized (manufactured, produced, etc.) by cells according to “blueprints” in the cellular DNA which are decoded by transcriptional processes to produce messenger RNA (mRNA) which are then translated into a protein. After translation, post-translational modifications may add or modify the resulting protein into a final form. Examples of post-translational modifications include folding modifications, changing or removing amino acids, adding/removing disulfide bridges, modifications to binding functions (e.g., glycosylation, prenylation, and acetylation), and adding functional groups (e.g., phosphorylation, nitrosylation, and GTP binding).

A desired protein may thus be synthesized by cells using a recombinant protein expression technique where the genetic blueprint for the desired protein is inserted as an expression vector into cells so that the cellular transcription process produces mRNA that is translated into the desired protein. Commonly used cells for recombinant protein expression systems include yeast (e.g., Pichia pastoris), bacteria (e.g., Escherichia coli), plant (e.g., one of the Nicotiana or Allium genus), mammalian, insect, and algal cells. In addition, cell-free protein expression may be used to in vitro synthesize proteins where the transcription, translation, and post-translational modifications are performed in vitro, for example, with cell extracts instead of cell cultures.

The rMBL may be encoded by DNA sequences encoding functionally equivalent variations of APA, which may be cloned and/or derived from naturally occurring allium porrum agglutinin. Functionally equivalent variations may arise naturally or by design from one or more of (a) slight variations in DNA sequences that do not alter the encoded protein, (b) slight variations in the protein and amino acid sequence that do not substantially alter the overall function (as may be measured by, for example, binding affinity for one or more pathogens), and (c) variations in which the protein and amino acid sequence are shortened (e.g., representing the effects of, for example, post-translational modifications). As used herein, the term “rMBL” encompasses such functionally equivalent variations of APA.

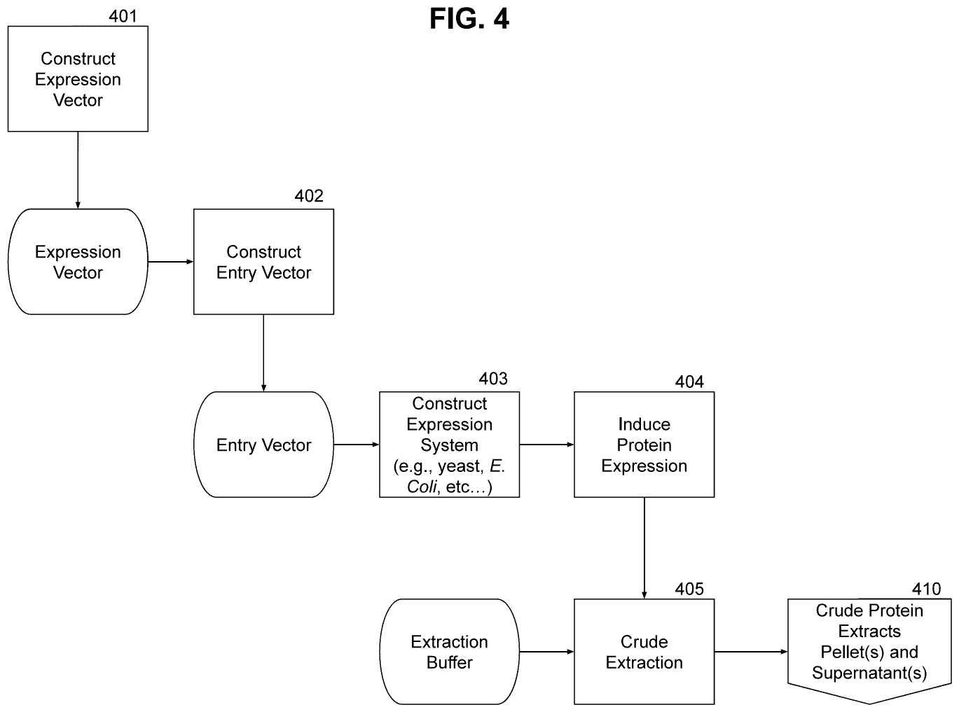

FIG. 4 illustrates an example rMBL expression process flow for yielding crude protein extracts. An expression vector was created in Construct Expression Vector step 401 where primers and tags (e.g., His, S and Trx tags) were prepended and appended to the desired MBL protein for expression by an expression system (e.g., Escherichia coli (E. coli) bacteria including BL21(DE3) and BL21-AI strains). A primer pair (including a forward primer and a reverse primer) was designed to amplify the MBL Open Reading Frame (ORF). The forward primer was 5′-GGGGACAAGTTTGTACAAAAAAGCAGGCTTAGAAAACCTGTATTTTCAGAGCATGG GCCGTACTACTCCA-3′, in which an attB1 recombinant sequence, an ATG start codon, and a short sequence encoding a TEV protease digestion site were included. As this forward primer was long, it was separated into into two overlapped primers, lectin-TEV-F1: 5′-GGGGACAAGTTTGTACAAAAAAGCAGGCTTAGAAAACCTG-3′ and lectin-TEV-F2: 5′-GCTTAGAAAACCTGTATTTTCAGAGCATGGGCCGTACTACTCCA-3′. The reverse primer was 5′-GGGGACCACTTTGTACAAGAAAGCTGGGTATCAAGCAGCACTGGTACCAAC-3′, which contains an attB2 recombinant sequence and a TCA stop codon.

For PCR (Polymerase Chain Reaction), 10 ng purified PUC-kan-lectin plasmid was used as template for the first step PCR with lectin-TEV-F2 and the reverse primer. The thermal cycle used consisted of 94° C. for 5 min, followed by 30 cycles of 94° C. for 30 s, 60° C. for 1 min, and 72° C. for 30 s. The final extension step was 10 min at 72° C. The resulting PCR products were diluted 100 times as template for the second PCR with lectin-TEV-F1 and reverse primers. The thermal cycle consisted of 94° C. for 5 min, followed by 30 cycles of 94° C. for 30 s, 60° C. for 1 min, and 72° C. for 30 s. The final extension step was 10 min at 72° C.

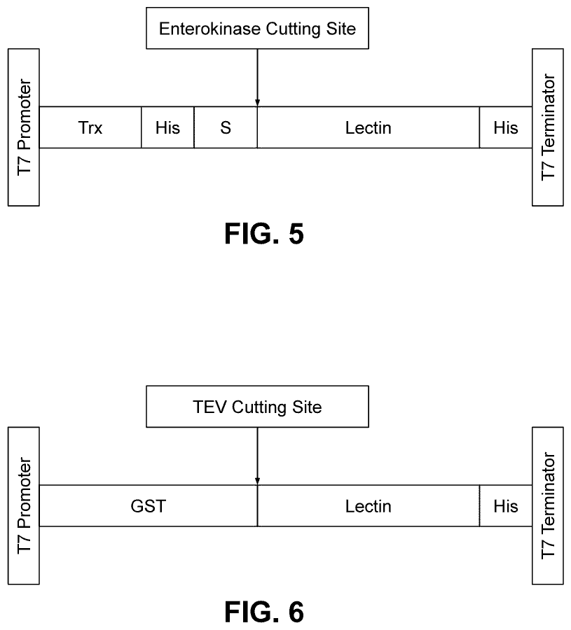

The second step PCR products were then ligated to the pDONR221 entry vector by using Gateway BP Clonase II Enzyme Mix (Invitrogen, USA) following the manufacturer's protocol at step 402. Colonies grown on solidified lysogeny broth (LB) medium (described below) were PCR screened for positive colonies which were used to amplify recombinant plasmids, identified as pDONR221-TEV-LEC. Purified pDONR221-TEV-LEC plasmids were used to clone rMBL ORF into expression vectors (including pET32a, identified as pET32a-Trx-Lec, and pDEST) in which the rMBL ORF was fused with a tag (e.g., Trx or GST, thioredoxin or glutathione S-transferase, respectively) and cutting site (e.g. enterokinase or TEV (Tobacco Etch Virus) protease cutting site) to the N-terminal end and a polyhistadine (His) tag to the C-terminal end (see FIG. 5 and FIG. 6, respectively).

The ligated products (shown as entry vector in FIG. 4) were introduced into TOP 10 E. coli competent cells according to the manufacturer's protocol 403. The pET32a-Trx-Lec expression vector was then introduced into chemically competent E. Coli cells including BL21(DE3) and BL21(AI) strains for rMBL induction, which were identified as pET32a-Trx-Lec/BL21(DE3) and pET32a-Trx-Lec/BL21-AI, respectively. A similar process for rMBL expression and induction of a GST tagged lectin was performed with pDEST15-GST-Lec.

At rMBL induction 404, the E. coli cells are incubated in an appropriate environment and induced into producing the rMBL. The E. coli are inoculated into a sterilized lysogeny broth (LB) medium (prepared by dissolving 10 g tryptone, 5 g yeast extract, and 10 g NaCl per liter of deionized water to which 7 g of agar could be added to solidify) with varying supplements for incubation and to induce rMBL production.

For some recombinant constructs (e.g., pET32a-Trx-Lec/BL21(DE3)pLysS), LB can be supplemented with 200 mg/L kanamycin and 34 mg/L chloramphenicol in a 50 ml tube which is placed on an incubator shaken at 200 rpm for 16 hours at 37° C. Then, 0.1 ml suspension culture from each tube was inoculated into another 10 ml of freshly prepared LB broth medium containing 200 mg/L ampicillin and 34 mg/L chloramphenicol in another 50 ml tube. These tubes were placed on an incubator shaken at 120 rpm at 37° C. for a few hours until the absorbance value of suspension culture measured at 600 nm was 0.7. Then, the isopropyl-1-thio-beta-D-galactopyranoside (IPTG) was added to the suspension cultures to a final concentration of 1 mM. After induction with IPTG at varying conditions (including 30° C. for 4 h, 20° C. for 20 h, and 37° C. for 4 h), the suspension cultures were harvested by centrifugation to obtain cell pellets for crude protein extraction.

For some recombinant constructs (e.g., pET32a-Trx-Lec/BL21(DE3)), LB can be supplemented with 100 mg/L ampicillin in a 50-ml tube which is placed on an incubator shaken at 200 rpm at 37° C. for 16 h. After the absorbance value of suspension culture reached to 1.0 measured at 600 nm on a spectrometer, 0.1 ml suspension culture was then inoculated to 10 ml of freshly prepared LB broth medium containing 100 mg/L ampicillin in another 50 ml tube. These tubes were placed on an incubator and shaken at 120 rpm at 37° C. until the absorbance value measured at 600 nm reached to 0.7. IPTG was added to the suspension culture in each tube to a final concentration of 1 mM. E. coli cultures were then harvested by centrifugation after 4 hours of continuous shaking.

For some recombinant constructs (e.g., pET32a-Trx-Lec/BL21(AI)), LB can be supplemented as described above for pET32a-Trx-Lec/BL21(DE3) with the following modification: in addition to adding IPTG, adding L-(+)-arabinose to the suspension cultures to a final concentration of 0.2%. E. coli cultures were then harvested by centrifugation after 4 hours of continuous shaking.

For all rMBL inductions, the harvested E. coli pellets were stored in a −80° C. freezer for protein extraction.

At crude protein extraction 405, an appropriate extraction buffer is used on the E. coli pellets. The extraction buffer may be built upon a base of 20 mM pH 8.0 Tris-HCl which may be an appropriate extraction buffer for GST-tagged rMBL. For extracting His-tagged rMBL, an appropriate extraction buffer may be composed of 20 mM pH 8.0 Tris-HCl supplemented with imidazole (e.g., at 10 or 20 mM). Urea may also be added to create a denaturing extraction buffer (e.g., 20 mM Tris-HCl pH 8.0, 8M urea, and 10 mM imidazole) to improve lysis and extraction. E. coli pellets were suspended in a 50 ml extraction buffer, to which lysozyme may be added to a final concentration of 0.1 mg/ml, for 30 min lysis at room temperature. The resulting mixture was placed on ice and sonicated at supersonic for 5 min to further lyse cells. The resulting lysate mixture was centrifuged at 12,000 rpm and 4° C. for 20 min. The supernatants and pellets containing crude proteins were transferred and then stored on ice for purification.

Regardless of the method used to express and extract the rMBL, the output of the rMBL expression step 405 is crude protein extracts 410, e.g. in pellets and supernatants forms, which includes the rMBL in addition to undesired elements consisting of byproducts from the recombinant expression process. A multi-stage purification process is required to remove undesired elements to produce a more pure and more concentrated solution of our rMBL.

### 5. rMBL Purification

Protein purification uses various techniques to remove any undesired elements from the expressed crude solution with the goal of having as much of the target protein remaining. After protein expression, it may be necessary to separate the target protein from the mixture (e.g., biological culture) by destroying the host cells via lysing (e.g., osmotic shock or ultrasonication) prior to downstream purification for removing undesirable elements (e.g., centrifugation).

When a cell is lysed, all of the intracellular contents, including the target protein, are released from the cell membrane. Therefore, when considering purification techniques, the properties of the target protein in addition to the other elements in the mixture (e.g., cellular waste and debris, which includes undesirable elements) were considered. Properties of these elements in the mixture which may be considered in designing a purification process include, but are not limited to: chemical, structural, and functional properties including size, shape, charge and isoelectric point (pI), solubility, precipitability, density, stability, hydrophobicity/hydrophilicity, and various binding affinities. Various purification methods may be employed to remove undesirable elements including (but not limited to) chromatography and separation techniques utilizing binding affinity, charge, size, and precipitation.

A purification process may include multiple purification techniques to optimize yield with consideration for throughput, purity, and costs. Multiple techniques are typically combined into an overall purification process to achieve a desired yield and purity level. While additional purification steps could lead to higher purity, each additional purification step could also reduce yield and/or increase costs. A non-exhaustive list of potential purification techniques are described below which may be applied as described or with similar techniques (e.g., a larger scale version of a process described here).

Lysis is the process of breaking open cells, which are typically responsible for expressing the target protein, to free the target protein for purification. There are various lysing methods which may be selected depending on the cell type, nature of the target protein, and downstream purification steps and applications. The lysing step chosen may preferably be non-destructive to the target protein preserving the target protein's structure, function, binding activity, and/or reconstitutability (e.g., refolding after denaturation). Common lysing methods include: mechanical disruption (e.g. sonication or homogenization), chemical disruption (e.g., lysis with a detergent or alkaline), enzymatic digestion (e.g., with a lysozyme or protease), freeze-thaw lysis, pressure-based lysis, electrical disruption, heat shock, and other commercially available lysis buffers.

In addition to cell lysis, temperature and temperature changes (e.g., cooling and/or heating) may be part of the purification process as freezing and boiling can each damage, denature, or otherwise destroy unwanted elements which may aid in extracting a target protein, especially if the target protein is thermostable or reconstitutable. Low temperatures (e.g., 4° C.) may be helpful for certain purification steps as it may reduce activity and/or increase stability.

Centrifugation is a method of separating molecules based on their varying densities by spinning them in a solution at high speed, creating a centrifugal force on the tube. Denser components will migrate away from the axis of the centrifuge to the bottom of the vial and precipitate (“pellet”), while less dense components in the supernatant remain closer to the axis. After centrifugation, the less dense supernatant may be separated from the denser precipitate. Varying the centrifugal force applied (e.g., magnitude and duration) varies the rate of separation.

In addition, centrifuges could incorporate centrifugal filters for filtering out materials at different molecular masses as measured by kilodaltons (kDa), for example. Common commercial filter molecular weight cutoffs (MWCO) include 3 kDa, 30 kDa, 50 kDa, and 100 kDa. A centrifugal filter may be chosen to separate proteins with the target protein in the supernatant or pellet as necessary for the overall purification process.

Chromatography is a lab-scale technique for separating molecules based on various properties which can be used for purifying a target protein. There are multiple chromatography techniques which can be utilized for separating the desired components from the undesired elements based on chemical properties (e.g., binding affinity) or physical properties (e.g., size inclusion and/or exclusion). Desirable components in a fluid mixture may be chromatographically separated using a stationary phase material to attract and hold the target protein while undesired components flow through. Afterwards, the target protein may be eluted (washed out) from the stationary phase material for collection. Less commonly, undesirable components may be attracted such that the desirable components flow through.

Affinity chromatography is a chemical property based purification technique that leverages the selective affinity between a receptor on the target protein and a ligand, usually on a solid stationary substrate. A compound containing tagged proteins (such as polyhistidine (histidine-tag/His-tag), glutathione S-transferase (GST), maltose-binding protein (MBP), Strep-tag® II, and FLAG™ affinity tags) may be purified using affinity chromatography where a corresponding ligand for the tag is used to bind the tagged proteins to the substrate. Using His-tagged proteins as an example, binding can be achieved via immobilized metal affinity chromatography (IMAC). In this case, the stationary IMAC might be composed of agarose beads with Ni-NTA (Ni is a nickel metal ion) as ligands for binding to His-tags. A mixture containing the tagged target protein can thus be purified using one or more tags as undesirable components that do not bind flow through and are washed out. The bound proteins may be detached from the ligand, for example with a competitive binding agent (e.g., imidazole in the case of His-tagged proteins).

Ion exchange chromatography is an example of a purification technique separating items based on their electrical or ionic charge. As proteins have some charge (either positive, negative, or neutral), they can be separated by their charge as opposites attract (i.e., negatively charged proteins are attracted to a positive charge and positively charged proteins are attracted to a negative charge). Anion-exchange chromatography may be used to attract negatively charged proteins while cation-exchange chromatography may be used to attract positively charged proteins. A mixture containing a charged target protein can thus be purified as undesirable components that are not sufficiently bound by electrostatic attraction will flow through and be washed out. The bound proteins may be detached by removing the applied charge or with a competitive binding agent.

Size-based purification techniques, including size exclusion chromatography, separate molecules, such as proteins, based on their size and/or molecular weight. A porous material may be used to separate larger molecules from smaller ones. For example, porous beads (e.g., commercially available Sephadex G-10, G-15, G-25 and G-50) in a column may be used which changes the travel time for molecules as they flow through the column slowed by the pores allowing fractionation of the column output for purified target protein. Depending on the size of the target protein, undesired molecules, and the available filters, size based purification techniques may slow the travel time of the target protein or, alternatively, slow the travel time for undesirable components.

Similarly, a porous or semi-permeable membrane may be used to separate a target protein from the crude mixture as, for example, when purifying with dialysis, which separates molecules through a semipermeable porous membrane (e.g., dialysis bag) by diffusion. Molecules smaller than the pore size may pass through the semipermeable membrane. Depending on the size of the target protein, undesired molecules, and the semipermeable porous membrane, the target protein may either remain or traverse the membrane.

Precipitation techniques, for example with salt or acetone, may be used to precipitate out a target protein (e.g., into a solid form such as a crystal or a powder) from a crude mixture. Salting out precipitates out the target proteins by the addition of salt which attracts water molecules away from charged proteins that become less soluble in the salt water. Similarly, acetone may be used to reduce the solubility of a target protein, causing it to precipitate out. After precipitation, centrifugation may be used to separate the precipitate. Some processes may also benefit from and optionally include salt (e.g., in LB or buffer). For example, wash buffers may contain 100-150 mM NaCl.

### 6. Example Purification Processes

A combination of the above techniques may be utilized in combination to purify rMBL from the crude protein extracts produced by the rMBL expression step 301 of FIG. 3.

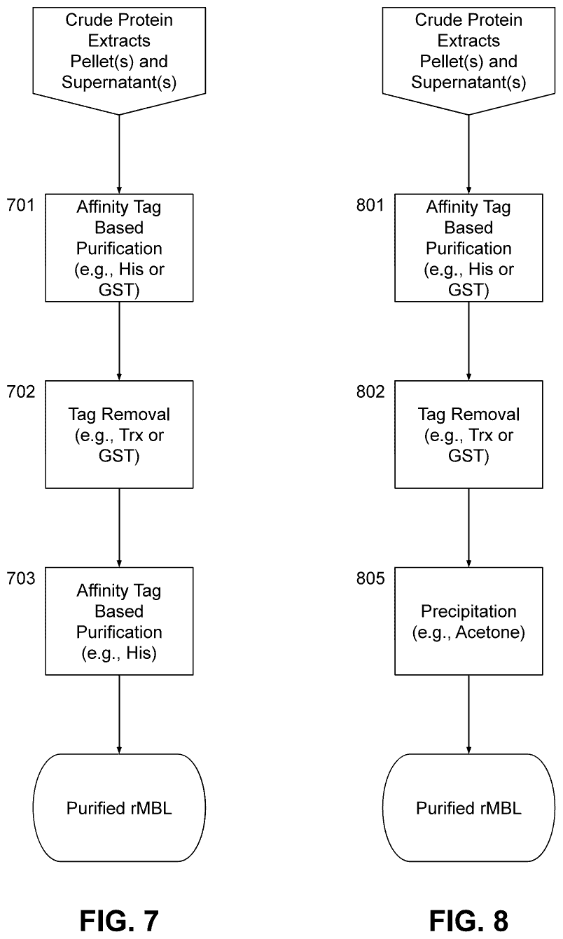

FIG. 7 illustrates a multi-step affinity tag based purification process. At 701, crude protein extracts are purified using an affinity tag (e.g., His or GST).

An example rMBL purification process of 701 includes utilizing a Ni-NTA column which performs immobilized metal affinity chromatography (IMAC) of recombinant proteins containing a His tag. A Ni-NTA column was prepared by loading one ml of Ni-NTA resin into a 10-ml syringe, stacked tightly via gravity, and washed three times with ten volumes of extraction or wash buffer (20 mM Tris-Cl pH 8.0, 20 mM imidazole, and optionally, 100 mM NaCl). Then, 10 ml of crude protein extracts is loaded onto the top of the Ni-NTA column and eluted by gravity at a flow rate of 0.5 ml/min. The Ni-NTA column was washed with an extraction or wash buffer to wash off non-binding proteins. An elution buffer (20 mM Tris-Cl pH 8.0, 250-500 mM imidazole) was used to elute Trx and His tagged rMBL from the column.

An example rMBL purification process of 701 includes utilizing a glutathione column which performs affinity chromatography of recombinant proteins containing a GST tag. A glutathione column was prepared by loading 1 ml of glutathione resin into a 10-ml syringe, stacked tightly via gravity, and washed three times with ten volumes of TBS buffer (Tris-Buffered Saline, pH7.4). Then, 10 ml of crude protein extracts is loaded onto the top of the column and eluted by gravity at a flow rate of 0.5 ml/min. The column was washed with a TBS buffer to wash off non-binding proteins. An elution buffer (TBS, 50 mM reduced glutathione) was used to elute GST tagged rMBL.

An example rMBL purification process of 701 includes utilizing a Ni-NTA column to purify recombinant proteins containing a His tag using a denaturing extraction buffer or wash buffer (e.g., 20 mM Tris-HCl pH 8.0, 8M urea, 10 mM imidazole, and optionally 100 mM NaCl). A Ni-NTA column was prepared as described above but washed three times with ten volumes of denaturing extraction buffer. Then, 10 ml of crude protein extracts is loaded onto the top of the Ni-NTA column and eluted by gravity at a flow rate of 0.5 ml/min. The column was washed with a denaturing extraction buffer to wash off non-binding proteins. In a cold room, the column was washed with a refolding buffer (20 mM Tris-Cl pH 8.0, 10 mM imidazole) to allow the rMBL to refold on the column. The refolded rMBL was eluted with an elution buffer (20 mM Tris-Cl pH 8.0, 250 mM imidazole).

At 702, an affinity tag (e.g., Trx or GST) may be removed.

An example rMBL purification process of 702 includes removing one or more tags (e.g., Trx tag) from the tagged rMBL. As shown by FIG. 5, the expressed rMBL includes a Trx, His, and S-tag prepended to the rMBL for purification. The S-tag, with an enterokinase cleavage site, enables enzymatic digestion by enterokinase to cut the prepended (Trx and His) tags from rMBL. The enterokinase enzymatic digestion was performed in 500 μl contained in 1.5 ml tubes. The reaction was composed of 25 mM Tris-HCl buffer (pH 8.0), 50 mM NaCl, 2 mM CaCl₂ , 300 μg partially purified rMBL, 175 mM imidazole (present in partially purified rMBL solution), and 50 units of enterokinase. The enzymatic digestion was carried out at 25° C. for 20 hours and 9.5 ml 20 mM Tris-HCl (pH 8.0) was added to dilute for further purification. After enterokinase digestion, the rMBL retains an appended His-tag for further purification using the Ni-NTA methods described above.

Similarly, an example rMBL purification process of 702 includes removing one or more tags (e.g., GST tag) from the GST tagged rMBL expressed with a prepended GST tag as shown in FIG. 6. A GST affinity tag can be removed with enzymatic digestion (e.g., with thrombin, factor Xa, or GE's PreScission Protease) or with a TEV protease if the GST tag is attached to the rMBL with a TEV cutting site. A glutathione column may be used to remove the severed GST tags and/or the remaining His-tag on the rMBL may be used for further purification using, for example, the Ni-NTA methods described above.

At 703, the remaining His-tag on the rMBL may be used for an additional purification as described above and, optionally, removing His-tag such as by using enzymatic cleavage. For example, a TEV protease may be used if the expression vector includes a TEV site between the His-tag and rMBL. An exopeptidase, such as a carboxypeptidase, may be used to remove C-terminal His-tags while aminopeptidases and endoproteases may be used to remove N-terminal His-tags. Alternatively, as the remaining His-tag is small and unobtrusive to the overall function of the rMBL, the remaining His-tag may not require removal.

A multi-step purification process is illustrated in FIG. 8 using an affinity tag and acetone precipitation. At 801, crude protein extracts are purified using an affinity tag (e.g., His or GST) as described above for 701. At 802, an affinity tag (e.g., Trx or GST) may be removed as described above for 702. An alternative purification process (not shown) may perform the acetone precipitation step 805 before tag removal step 802.

An example rMBL purification process of 805 includes acetone precipitation of the tagged rMBL to yield purified rMBL. Acetone precipitation may be performed by adding cold (e.g., −20° C.) acetone to the mixture (e.g., a volume of acetone up to approximately 4 times the volume of the rMBL containing mixture) followed by centrifugation (e.g., at 10,000-20,000 g for 10-30 minutes) to yield purified rMBL pellets. An example acetone precipitation process may include adding −20° C. acetone to a rMBL mixture at a 4:1 ratio and centrifuging the mixture at 15,500 g for 30 minutes.

### 7. Other Aspects of Disclosure

As a person skilled in the art will recognize from the previous detailed description and from the figures and claims, modifications and changes can be made to the composition, method, and/or variants without departing from the scope defined in the following claims.

1. A method of purifying a recombinant plant-derived mannose binding lectin composition comprising:
obtaining a first mixture (410) containing the recombinant mannose binding lectin which includes a first affinity tag and a second affinity tag that is different from the first affinity tag;
separating the first mixture (701) based on the first affinity tag to obtain a second mixture;
removing one of the first affinity tag or the second affinity tag (702) from the recombinant mannose binding lectin in the second mixture; and
separating the second mixture (703) based on the remaining affinity tag to obtain a third mixture.

2. The method of claim 1 wherein the recombinant mannose binding lectin is a recombinant allium porrum agglutinin.

3. The method of claim 1 where the first affinity tag is a His or GST tag.

4. The method of claim 1 where the second affinity tag is a GST or Trx tag.

5. The method of claim 1 wherein one or more of the separating steps use an affinity-based technique.

6. The method of claim 1 wherein the third mixture contains a higher concentration/proportion of recombinant mannose binding lectin than the second mixture, and the second mixture contains a higher concentration/proportion of recombinant mannose binding lectin than the first mixture.

7. The method of claim 1 further comprising separating based on size or molecular weight.

8. The method of claim 1 further comprising precipitating the recombinant mannose binding lectin.

9. The method of claim 8 wherein precipitating the recombinant mannose binding lectin comprises using acetone.

10. The method of claim 1 wherein obtaining the first mixture comprises using a recombinant expression system.

11. The method of claim 10 wherein the recombinant expression system uses bacteria, yeast, or a plant.

12. The method of claim 11 wherein the recombinant expression system uses Escherichia coli, Pichia pastoris, or a tobacco plant of the Nicotiana species.

13. The method of claim 10 wherein obtaining the first mixture further comprises expressing by the recombinant expression system a recombinant mannose binding lectin with the first affinity tag and the second affinity tag.

14. A method of purifying a recombinant plant-derived mannose binding lectin composition comprising:
obtaining a first mixture (410) containing the recombinant mannose binding lectin which includes at least a first affinity tag;
separating the first mixture (801) based on the first affinity tag;
precipitating the recombinant mannose binding lectin (805).

15. The method of claim 14 wherein the recombinant mannose binding lectin is a recombinant allium porrum agglutinin.

16. The method of claim 14 wherein precipitating the recombinant mannose binding lectin comprises using acetone.

17. The method of claim 14 wherein obtaining the first mixture comprises using a recombinant expression system.

18. The method of claim 17 wherein the recombinant expression system uses E. coli.

19. The method of claim 17 wherein obtaining the first mixture further comprises expressing by the recombinant expression system a recombinant mannose binding lectin with the first affinity tag and the second affinity tag.
